(19) United States
(12) Patent Application Publication
(10) Pub. No.: US 2026/0007546 A1
(43) Pub. Date: Jan. 8, 2026

(54) OCULAR IMPLANT, KIT FOR DEPLOYING IMPLANT, METHOD OF DEPLOYING IMPLANT

(71) Applicant: Oxford University Innovation Limited, Oxford (GB)

(72) Inventors: Yunlan ZHANG, Oxford (Oxfordshire) (GB); Yunfang YANG, Oxford (Oxfordshire) (GB); Jared CHING, Oxford (Oxfordshire) (GB); Zhong YOU, Oxford (Oxfordshire) (GB)

(21) Appl. No.: 18/881,749

(22) PCT Filed: Jul. 10, 2023
(86) PCT No.: PCT/GB2023/051812

§ 371 Date: Jan. 7, 2025

(30) Foreign Application Priority Data
Jul. 12, 2022 (GB) 2210218.0

(51) Int. Cl. A61F 9/007

(52) U.S. Cl. CPC A61F 9/00781; A61F 2250/001

(57) ABSTRACT

An ocular implants, kits and deployment methods are disclosed. In one arrangement, an ocular implant has an elongate body configured to be deployable at a deployment position at which the body extends from the anterior chamber of an eye to the subconjunctival space of the eye. The body comprises a conduit structure defining a conduit for promoting flow of aqueous humour through the conduit from the anterior chamber to the subconjunctival space. The body comprises an anchoring structure configured to expand from a radially contracted state to a radially expanded state.

The present disclosure relates to an ocular implant and associated deployment methods and kits.

Glaucoma is a leading cause of irreversible blindness and the leading cause of blindness globally. Open-angle glaucoma (POAG) is a common type of high-pressure glaucoma thought to be caused by a disruption in the drainage pathways of the eye. POAG can develop progressively over a number of years. Current treatment options for POAG focus on lowering intraocular pressure (IOP), which can be achieved by pharmacological topical medication (e.g., drugs delivered as eye drops), laser treatment, or surgical interventions.

Minimally invasive glaucoma surgery (MIGS) has emerged as a new treatment option in recent years. MIGS has become popular for its improved safety profile compared to traditional glaucoma drainage surgery. MIGS implants can be implanted using ab-interno or ab-externo approaches and involve little or no scleral dissection and minimal or no conjunctival manipulation.

MIGS implants have been demonstrated that effectively form artificial drainage routes for aqueous humour outflow and significantly reduce IOP. However, various shortcomings have been found with existing techniques, including a tendency for devices to migrate, and/or contribute to endothelial cell loss and/or hypotony.

It is an object of the present disclosure to provide improved implants and associated devices and methods for treating glaucoma.

According to an aspect of the invention, there is provided an ocular implant comprising: an elongate body configured to be deployable at a deployment position at which the body extends from the anterior chamber of an eye to the subconjunctival space of the eye, wherein: the body comprises a conduit structure defining a conduit for promoting flow of aqueous humour through the conduit from the anterior chamber to the subconjunctival space; and the body comprises an anchoring structure configured to expand from a radially contracted state to a radially expanded state.

Thus, an implant is provided that can adopt a radially contracted state to facilitate efficient insertion to a deployment position extending from the anterior chamber to the subconjunctival space, and which has an anchoring structure that can expand to a radially expanded state when the deployment position is reached. The conduit structure promotes flow of aqueous humour that can reduce IOP and the anchoring structure provides long-term positional stability and promotes bleb formation.

In an embodiment, the body is manufacturable by a manufacturing process comprising removing material from a hollow cylindrical tube in a region corresponding to the conduit structure and/or in a region corresponding to the anchoring structure. This approach allows the body to be manufactured efficiently and with high precision and yield.

In an embodiment, the conduit structure has a generally cylindrical form in a relaxed state and defines one or more lateral openings configured to facilitate bending of the conduit structure about axes perpendicular to a longitudinal axis of the conduit structure. This approach allows the flexibility of the conduit structure to be defined with high precision during the manufacturing process. Controlling the flexibility of the conduit structure is desirable to allow the conduit structure to have a bending stiffness that is similar to or lower than a bending stiffness of tissue along a deployment route (e.g., through the scleral channel) and/or in the region of the deployment position, such that the conduit structure can be forced by the tissue to flex into a shape that conforms with the tissue. This allows the body to be deployed reliably and with minimal risk of discomfort, irritation/injury, and/or extrusion.

In an embodiment, the anchoring structure comprises a plurality of arms, at least a subset of which extend along paths that each lie within a different respective plane containing a longitudinal axis of the anchoring structure. Configuring the anchoring structure to have arms arranged in this manner has been found to promote bleb formation efficiently. The alignment of the arms within the planes containing the longitudinal axis facilitates manufacture and injury free insertion of the implant.

In an embodiment, the arms are arranged in a rotationally asymmetric manner to promote rotationally asymmetric pushing away of tissue in the region of the anchoring structure when the body is deployed at the deployment position. Configuring the anchoring structure to be rotationally asymmetric may facilitate positioning of the anchoring structure in a rotational orientation that enhances bleb formation, for example with arms that extend radially to a greater extent in a direction towards subconjunctival tissue to be supported to promote bleb formation.

In an embodiment, at least two, optionally all, of the arms converge and join at or near a distal tip of the anchoring structure. Arranging for the arms to reconnect in this way provides structural strength to the anchoring structure and avoids having exposed arm ends that might puncture or irritate tissue or limit a safe range of arm stiffnesses that can be provided.

In an embodiment, at least two, optionally all, of the arms are disconnected from each other at distal extremities of the arms. Arranging for the arms to remain free at their distal ends provides greater freedom for shaping the arms to promote bleb formation and/or reduces interference to flow of aqueous humour after it has left the conduit of the conduit structure.

In an embodiment, the body is configured such that when the body is in a relaxed state before deployment a longitudinal axis of the conduit structure is aligned obliquely with respect to a longitudinal axis of the anchoring structure. Arranging for the conduit structure to be obliquely aligned with the anchoring structure in this way may facilitate conformity between the body and surrounding tissue at the deployment position.

In an embodiment, the implant comprises a biodissolvable material on or in the body, the biodissolvable material being configured to dissolve progressively over a period of time after deployment and to thereby gradually alter an effect of the implant on flow of aqueous humour through the conduit defined by the conduit structure from the anterior chamber to the subconjunctival space. The gradual alteration may, for example, comprise a gradual increase in the flow (i.e., a gradual decrease in outflow resistance). Configuring the implant to cause the outflow to increase gradually in this way (rather than suddenly) may decrease the risk of early hypotony.

Embodiments of the disclosure will now be further described, merely by way of example, with reference to the accompanying drawings.

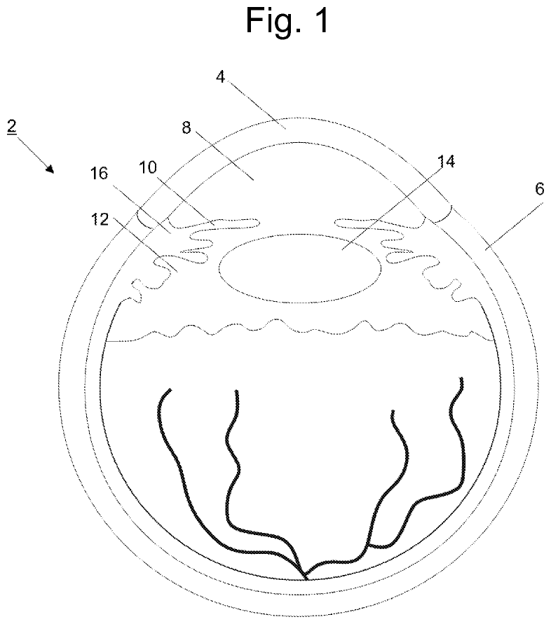

FIG. 1 is a cross-sectional view of a portion of an eye.

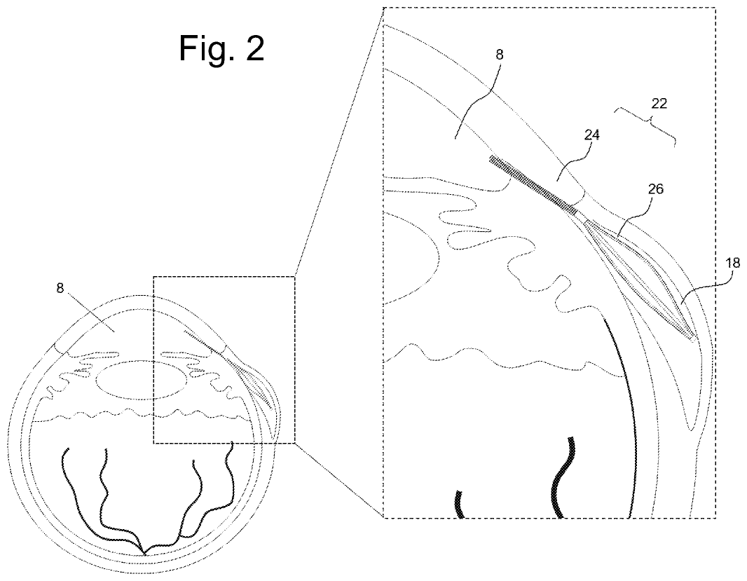

FIG. 2 is an enlarged cross-sectional view of a portion of an eye showing positioning of an elongate body of an ocular implant extending from an anterior chamber to a subconjunctival space.

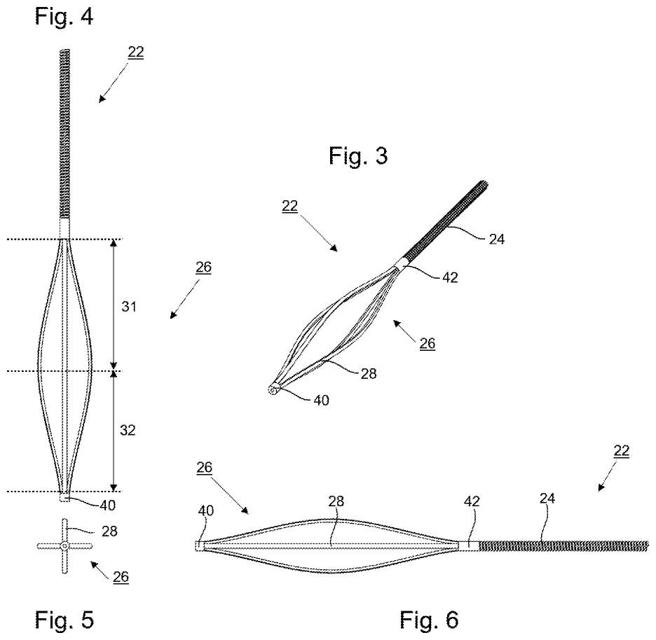

FIG. 3 is a perspective view of an elongate body of an example implant in which all arms of an anchoring structure have the same shape and are joined together at a distal tip.

FIG. 4 is a top view of the elongate body of FIG. 3.

FIG. 5 is an end view of the elongate body of FIG. 3.

FIG. 6 is a side view of the elongate body of FIG. 3.

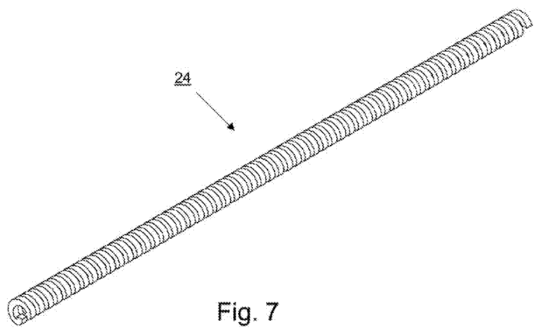

FIG. 7 is an enlarged view of a conduit structure of the elongate body of FIGS. 3-6.

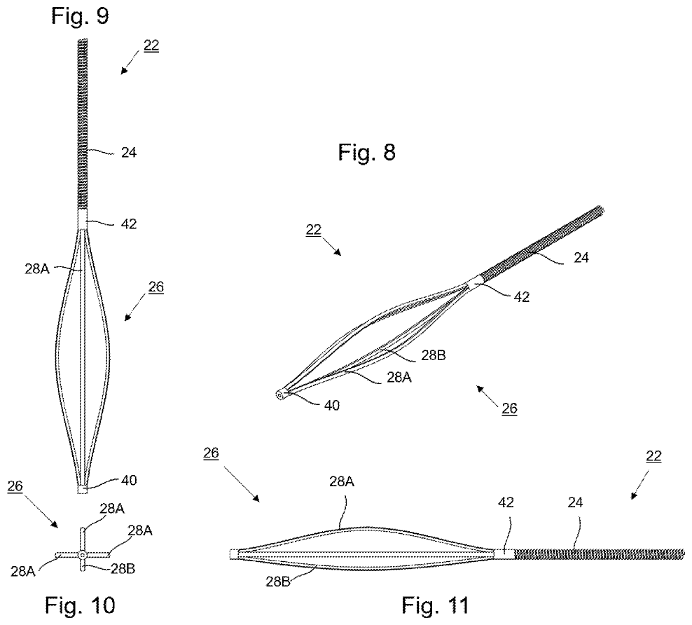

FIG. 8 is a perspective view of a variation on the arrangement of FIGS. 3 to 7 in which one of the arms of the anchoring structure radially diverges less than the other three arms for positioning on the sclera while the three more divergent arms support the conjunctiva.

FIG. 9 is a top view of the elongate body of FIG. 8.

FIG. 10 is an end view of the elongate body of FIG. 8.

FIG. 11 is a side view of the elongate body of FIG. 8.

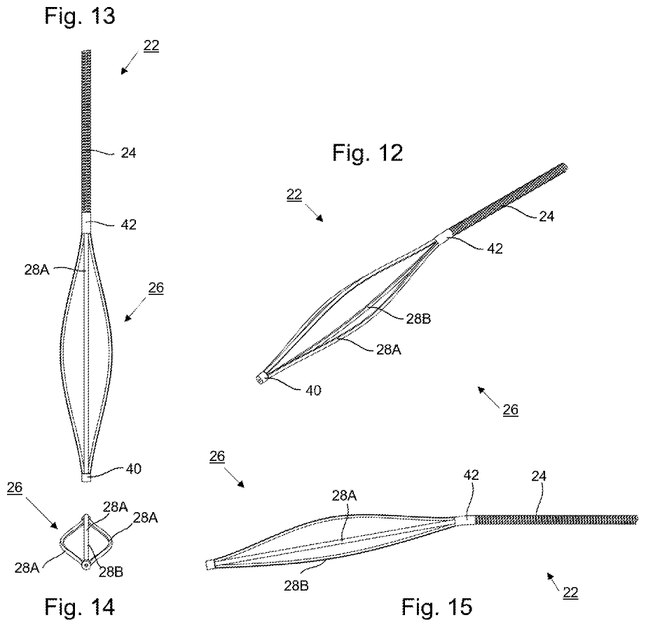

FIG. 12 is a perspective view of a variation on the arrangement of FIGS. 8 to 11 in which longitudinal axes of the conduit structure and anchoring structure are non-parallel.

FIG. 13 is a top view of the elongate body of FIG. 12.

FIG. 14 is an end view of the elongate body of FIG. 12.

FIG. 15 is a side view of the elongate body of FIG. 12.

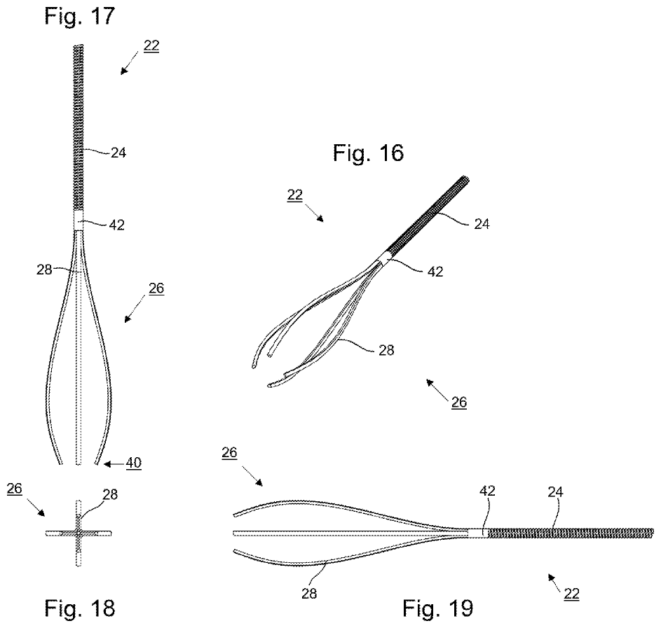

FIG. 16 is a perspective view of a variation on the arrangement of FIGS. 3 to 7 in which the arms are disconnected from each other at the distal tip.

FIG. 17 is a top view of the elongate body of FIG. 16.

FIG. 18 is an end view of the elongate body of FIG. 16.

FIG. 19 is a side view of the elongate body of FIG. 16.

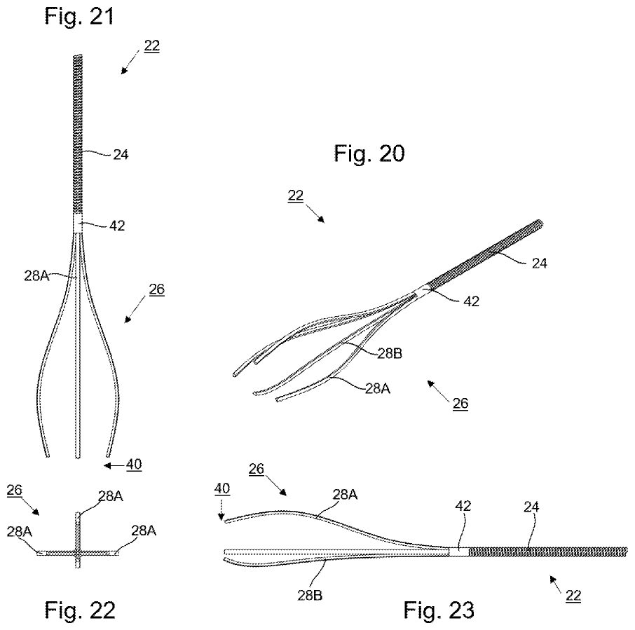

FIG. 20 is a perspective view of a variation on the arrangement of FIGS. 8 to 11 in which the arms are disconnected from each other at the distal tip.

FIG. 21 is a top view of the elongate body of FIG. 20.

FIG. 22 is an end view of the elongate body of FIG. 20.

FIG. 23 is a side view of the elongate body of FIG. 20.

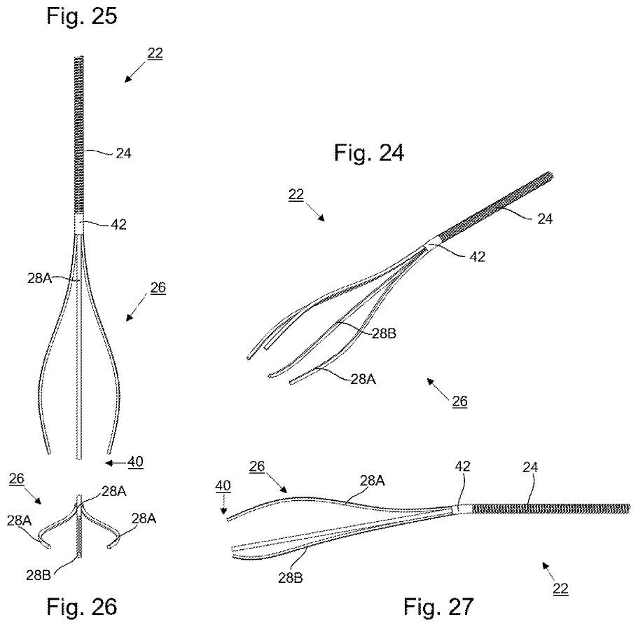

FIG. 24 is a perspective view of a variation on the arrangement of FIGS. 12 to 15 in which the arms are disconnected from each other at the distal tip.

FIG. 25 is a top view of the elongate body of FIG. 24.

FIG. 26 is an end view of the elongate body of FIG. 24.

FIG. 27 is a side view of the elongate body of FIG. 24.

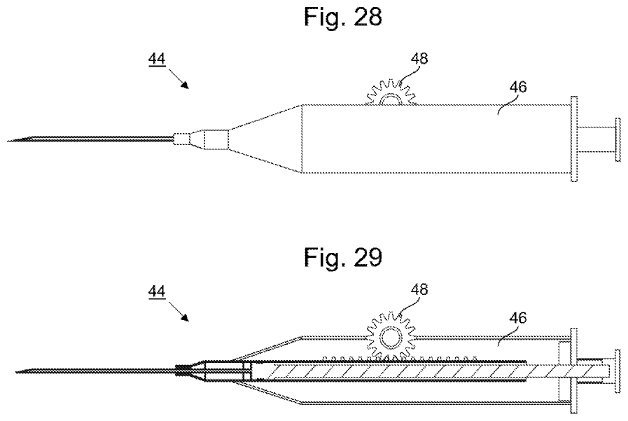

FIG. 28 is a side view of a delivery system for deploying an implant into the eye.

FIG. 29 is a sectional view of the delivery system of FIG. 28.

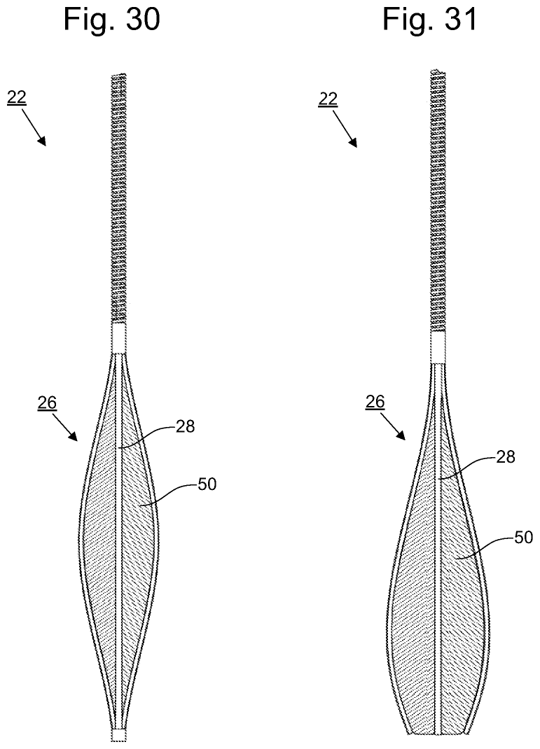

FIG. 30 shows a variation of the arrangement of FIG. 9 in which a biodissolvable material is provided as a coating over the anchoring structure.

FIG. 31 shows a variation of the arrangement of FIG. 17 in which a biodissolvable material is provided as a coating over the anchoring structure.

FIG. 1 is a sectional perspective view showing a portion of a human eye 2. A human eye is approximately a sphere 2.5 cm in diameter. The cornea 4 forms around one-sixth of the globe's circumference, and the sclera 6 forms the remaining five-sixths. Anterior chamber 8 is the aqueous humour filled space between the iris 10 and cornea 4. The posterior chamber 12 is a narrow space posterior to the iris 10 and anterior to the lens 14. Humans view an object through the cornea 4, the aqueous humour, and the lens 14.

Aqueous humour is a transparent fluid similar to plasma that maintains the intraocular pressure of the eyes, provides nutrition for the avascular ocular tissues, and remove wastes from these tissues. The aqueous humour is secreted by the epithelium from the ciliary body 16 and fills both anterior and posterior chambers. In a healthy eye, a stream of aqueous humour flows out of the eye as the ciliary body 16 secretes new aqueous humour. Most of the excess aqueous humour (70%-90%) leaves the anterior chamber through the trabecular meshwork and Schlemm's canal. The canal is drained by 25-35collector channels and between two and eight aqueous veins. A proportion (10%-30%) of aqueous humour drains via the suprachoroidal space. If these natural drainage channels are clogged, the eye pressure rises. Glaucoma is a condition where the optic nerve that connects the eye to the brain becomes damaged under such high intraocular pressure (IOP).

Implants can be used to increase aqueous outflow and avoid dangerous IOPs but existing implants have been found to have various shortcomings as mentioned in the introductory part of the description. Embodiments of the present disclosure aim to at least partially address one or more of these shortcomings.

Referring to FIG. 2, ocular implants are disclosed that comprise an elongate body 22. The body 22 is configured to be deployable at a deployment position in the eye. The body 22 comprises a conduit structure 24 and an anchoring structure 26. An example body 22 deployed at the deployment position is shown in FIG. 2. The body 22 extends from the anterior chamber 8 to the subconjunctival space 18 when at the deployment position. The conduit structure 24 defines a conduit for promoting flow of aqueous humour through the conduit from the anterior chamber 8 to the subconjunctival space 18. The deployment position may be such that the conduit structure 24 extends by a small amount into the anterior chamber 8 when the body 22 is at the deployment position, such as by about 1 mm. The body 22 may be coated by a biocompatible coating (e.g., silicone, parylene C, Poly(Styrene-block-IsoButylene-block-Styrene)—“SIBS”, etc.).

The anchoring structure 26 is configured to expand from a radially contracted state to a radially expanded state. The anchoring structure 26 may, for example, expand from the radially contracted state to the radially expanded state substantially without expansion of the conduit structure 24. The expansion of the anchoring structure 26 may thus occur independently of the conduit structure 24. The anchoring structure 26 may be configured to self-expand and/or be caused to expand by application of a stimulus, optionally by balloon actuation, hydraulic actuation, temperature actuation, or magnetic actuation.

The anchoring structure 26 may provide an anchoring force that resists longitudinal movement of the body 22 as a whole when in the radially expanded state. The anchoring structure 26 thus provides positional stability and helps to ensure that the body 22 reliably remains at a desired deployment position after deployment. In some embodiments, the anchoring structure 26 may be configured to promote formation of a bleb in the subconjunctival space 18 when the body 22 is deployed at the deployment position and the anchoring structure 26 is in the radially expanded state. This functionality is persistent, and the resulting bleb may be referred to as a long-term subconjunctival bleb. The anchoring structure 26 may, for example, be configured to have sufficient stiffness and appropriate geometry to adapt (e.g., push back) tissue in the subconjunctival space to promote bleb formation. The anchoring structure 26 may push tissue away from an axis of the anchoring structure 26, thereby holding tissue apart in the region of deployment. The anchoring structure 26 may have a maximum radial diameter in the range of about 0.2 mm to about 3.5 mm, preferably about 0.75 mm to about 2.0 mm, when deployed at the deployment position in the radially expanded state. These size ranges have been found suitable to provide an appropriate balance between expanding the subconjunctival space to promote bleb formation and avoiding negative effects such as hypotony, retinal detachments, or excess tissue damage.

In some embodiments, the body 22 is configured to be insertable to the deployment position while the anchoring structure 26 in the radially contracted state by applying a radially constraining force to the body 22 during the insertion. This facilitates insertion of the body 22 to the deployment position and/or minimizes a risk and/or extent of discomfort and/or injury. For example, the anchoring structure 26 may be configured such that the body 22 can fit inside a 25-27G delivery needle when the anchoring structure 26 is in the radially contracted state. The body 22 can be deployed at the deployment position by releasing the radially constraining force applied to the body 22 to allow the anchoring structure 26 to self-expand. Thus, once the body 22 is positioned as desired, the body 22 can effectively be fixed in place by allowing the anchoring structure 26 to expand and thereby resist unwanted longitudinal movement away from the desired deployment position.

In some embodiments, the conduit defined by the conduit structure 24 has a substantially constant cross-sectional area along a length of the conduit. The conduit may be substantially cylindrical for example. A cross-sectional area and length of the conduit and/or a wall thickness of the conduit structure 24 may be selected to control the outflow resistance when deployed such that it is low enough to treat high IOP but not so low that hypotony might occur. The conduit may typically be configured to have a outflow resistance that can control (e.g., reduce) IOP to be maintained in the range of, for example about 4 to 20 mm Hg, preferably in the range of about 6 to 12 mm Hg. The conduit structure 24 may be designed for example using Hagen-Poiseuille law to achieve an appropriate outflow resistance.

In some embodiments, the conduit structure 24 has a generally cylindrical form in a relaxed state (e.g., prior to deployment, when no external forces are being applied to the body 22). The conduit structure 24 may define one or more lateral openings that facilitate bending of the conduit structure 24 about axes perpendicular to a longitudinal axis of the conduit structure 24. The lateral openings may be referred to as cut-outs, cut patterns or radial openings. The lateral openings may be provided in wide range of patterns (e.g., lattices, pores, etc.) but preferably are arranged such that a resulting stiffness of the conduit structure 24 is comparable to or smaller than tissue at the intended deployment position. In one example arrangement, as shown in FIG. 7, the conduit structure 24 may be formed by a spiral (i.e., with lateral openings defining the spiral). Alternatively or additionally, the lateral openings may form multiple individual cells to define a lattice structure. The lateral openings may define a porous structure. The conduit structure 24 may thus be made relatively flexible. The conduit structure 24 may be able to flex through angles that allow the conduit structure 24 to adapt to the surrounding tissues, and connect the anterior chamber to the subconjunctival space while maintaining a fluidically continuous conduit along the length of the conduit structure 24. For example, the conduit structure 24 may be configured to be able to flex through angles of up to at least 20 degrees, optionally at least 30 degrees, optionally at least 45 degrees, while maintaining a fluidically continuously conduit along the length of the conduit structure 24. In some embodiments, the bending stiffness of the conduit structure 24 is selected to be similar or lower than a bending stiffness of tissue along a deployment route (e.g., through the scleral channel) and/or in the region of the deployment position, such that the conduit structure 24 can be forced by the tissue to flex into a shape that conforms with the tissue. Configuring the conduit structure 24 to be flexible in this manner allows the body 22 to be deployed reliably and with minimal risk of discomfort, irritation/injury, and/or extrusion. The conduit structure 24 can, for example, flex to follow curvature of the scleral channel during insertion.

A portion 42 of the conduit structure 24 adjacent to the anchoring structure 26 may be configured to have fewer lateral openings in comparison with regions of the conduit structure 24 positioned further from the anchoring structure 26, or the portion 42 may be configured to have no lateral openings at all for a portion of the length of the conduit structure 24 (as exemplified by the embodiments shown in FIGS. 3-27). This is because bending moments may tend to be higher in this region and reducing or avoiding lateral openings in the portion 42 may reduce or avoid risk of material failure in this region. As described below, in some embodiments a deliberate bend (oblique angling) is provided between the conduit structure 24 and the anchoring structure 26 and the higher strength provided in portion 42 may be used to maintain or support the bend.

The body 22 may be manufacturable by a manufacturing process comprising removing material from a hollow cylindrical tube. The material may be removed by laser etching for example. Material may be removed in a region corresponding to the conduit structure 24 and/or in a region corresponding to the anchoring structure 26. The removal of material from the region corresponding to the conduit structure 24 may increases a flexibility of the conduit structure 24 (e.g., to provide one or more lateral openings that facilitate bending about axes perpendicular to the longitudinal axis of the conduit structure 24 as mentioned above). The manufacturing process may comprise deforming the tube in the region corresponding to the anchoring structure 26 after the removal of material in the region. The deformation may define the anchoring structure 26 and configure the anchoring structure 26 to self-expand from the radially contracted state to the radially expanded state. Other manufacturing techniques may also be used, such as additive manufacturing.

In some embodiments, as exemplified in FIGS. 3-7, 8-11, 12-15, 16-19, 20-23, and 24-27, the anchoring structure 26 may comprise a plurality of arms 28. The arms 28 may extend generally in the longitudinal direction while radially diverging and/or converging. At least a subset of the plurality of arms may extend along paths that each lie within a different respective plane containing a longitudinal axis of the anchoring structure (thus providing arms that extend along paths having axially aligned components). Configuring the anchoring structure 26 with such arms 28 has been found to promote bleb formation efficiently, while being easily manufacturable. The axial alignment of the arms facilitates insertion of the implant with minimal risk of irritation/injury. The arms 28 may be formed by laser etching longitudinally extending openings in a region of a cylindrical tube and longitudinally compressing that region of the tube. The arms 28 are configured such that when the anchoring structure 26 is in the radially expanded state, as exemplified in the embodiments of FIGS. 3-27, the arms radially diverge as a function of position towards a distal tip 40 of the body 22 along at least a portion of the longitudinal axis of the anchoring structure 26. As indicated in FIG. 4, for example, the radial divergence of the arms may occur along a first portion 31 of the longitudinal axis of the anchoring structure 26. In some embodiments, the plurality of arms 28 may radially converge as a function of position towards the distal tip 40 of the body 22 along at least a portion of the longitudinal axis of the anchoring structure 26, for example along a second portion 32 of the longitudinal axis of the anchoring structure 26 as depicted in FIG. 4. The first portion 31 is between the conduit structure 24 (and, where present, portion 42) and the second portion 32.

In some embodiments, as exemplified in FIGS. 3-7, a maximum radial divergence of the arms 28 is the same. This can be seen most easily in the end view of FIG. 5. In this embodiment, the anchoring structure 26 has four arms 28 and each of the four arms extends radially outwards by the same amount. In this example all of the arms 28 have the same profile along the whole longitudinal length of the anchoring structure 26. The anchoring structure 26 has rotational symmetry about the longitudinal axis of order four. In other embodiments, the anchoring structure 26 may have higher or lower symmetry. For example, in some embodiments, the anchoring structure 26 may have rotational symmetry of order two or three or of an order higher than four.

In some embodiments, the anchoring structure 26 is configured to be rotationally asymmetric about the longitudinal axis (e.g., to have a rotational symmetry of order one). The rotational asymmetry may promote rotationally asymmetric pushing away of tissue in the region of the anchoring structure when the body is deployed at the deployment position. Configuring the anchoring structure 26 to be rotationally asymmetric may facilitate positioning of the anchoring structure 26 in a rotational orientation that enhances bleb formation. For example, when viewed along the longitudinal direction, the anchoring structure may be configured to extend radially to a greater extent on one side (e.g., through 180 degrees) than on the other side (e.g., through the other 180 degrees), which may be referred to as being a relatively flat side. This configuration may desirably be oriented when the body 22 is at the deployment position such that the relatively flat side is adjacent to the sclera and the more radially extending side is positioned opposite and acts to support the subconjunctival tissue and efficiently promote formation of a long-term bleb.

In some embodiments, as exemplified in FIGS. 8-11, 12-15, 20-23, and 24-27, at least two of the arms 28 have different maximum radial divergences. For example, two or more of the arms 28A may have the same maximum radial divergence and a single one of the arms 28B may have a lower maximum radial divergence. In the examples referred to above, the anchoring structure 26 has four arms, with three arms 28A having the same maximum radial divergence and one arm 28B having a smaller maximum radial divergence. In this case, arm 28B having the smaller maximum radial divergence would define the relatively flat side of the anchoring structure 26 and desirably positioned adjacent to the sclera while the other three arms 28A would support the subconjunctival tissue and efficiently promote formation of a long-term bleb.

In some embodiments, as exemplified in FIGS. 3-27, the plurality of arms 28 may comprise a first pair of arms extending along paths that lie in a same first plane and a second pair of arms extending along paths that lie in a same second plane, the first and second planes optionally being orthogonal to each other. In some embodiments, one or more of the pairs of arms in a same plane are arranged mirror symmetrically with respect to each other.

In some embodiments, as exemplified in FIGS. 3-7, 8-11, and 12-15, at least two, optionally all, of the arms converge and join (connect to each other) at or near a distal tip of the anchoring structure 26. In the examples shown, all four of the arms 28 join at the distal tip 40. Arranging for the arms 28 to reconnect in this way provides structural strength to the anchoring structure 26 and avoids having exposed arm ends that might puncture or irritate tissue or limit a safe range of arm stiffnesses that can be provided.

By contrast, in some embodiments, as exemplified in FIGS. 16-19, 20-23, and 24-27, at least two of the arms 28 are disconnected from each other (i.e., are free) at distal extremities of the arms (at or near the distal tip 40 of the anchoring structure 26). Arranging for some of the arms 28 to be free at their distal ends provides greater freedom for shaping the arms to promote bleb formation and/or reduces interference to flow of aqueous humour after it has left the conduit of the conduit structure 24. Increased anchoring forces are achievable for a given arm stiffness.

In some embodiments, as exemplified in FIGS. 3-7, 8-11, 16-19, and 20-23, the body 22 may be configured such that when the body 22 is in a relaxed state before deployment (i.e., with no significant external forces applied to the body 22) a longitudinal axis of the conduit structure 24 is coaxial with a longitudinal axis of the anchoring structure 26. Arranging for the conduit structure 24 to be axially aligned with the anchoring structure 26 in this way may facilitate insertion of the body 22 to the deployment position and/or manufacture of the body 22.

By contrast, in some embodiments, as exemplified in FIGS. 12-15, and 24-27, the body 22 may be configured (e.g., by a bend in portion 42 as mentioned above) such that when the body 22 is in the relaxed state before deployment a longitudinal axis of the conduit structure is aligned obliquely with respect to the longitudinal axis of the anchoring structure 26. The portion 42 may be stronger than surrounding portions of the body (even when the different portions of the body are manufactured from the same starting tube) due to having fewer lateral openings, which allows the portion 42 to support the bend and thereby maintain the oblique relative alignment. Arranging for the conduit structure 24 to be obliquely aligned with the anchoring structure 26 in this way may facilitate conformity between the body 22 and surrounding tissue at the deployment position.

The body 22 can be fabricated from various biocompatible materials with dimensions selected to provide the desired structural and mechanical attributes. Metallic or non-metallic materials may be used. Example materials include stainless steel, cobalt-chromium, tantalum, titanium, nitinol, and shape memory polymers, or biodesolvable materials. In some embodiments, the body 22 may be provided with a biocompatible coating that improves anchorage performance and/or antifibrotic properties. Alternatively or additionally, the body 22 may include a therapeutic agent supported by the body 22. The therapeutic agent may be incorporated into a polymeric coating that is deposited on an outer and/or inner surface of the body 22. In some embodiments, the therapeutic agent comprises an anti-glaucoma drug and/or a biodegradable drug matrix. Examples of anti-glaucoma drugs include prostaglandin analogues. In some embodiments, the implant comprises a biocompatible coating on or in the body that modifies a flow of aqueous humour to improve IOP regulation (e.g., by modifying an outflow resistance) and/or modifies a stiffness of the body or a portion of the body. The coating may form an inner and/or outer layer of the body. The coating may be configured to be drug-eluting.

In an embodiment, a kit is provided for deploying the implant into the eye. The kit may comprise an implant according to any of the embodiments discussed herein. As exemplified in FIGS. 28 and 29, the kit may further comprise a delivery system 44 configured to deliver the implant to the deployment position. The implant may be provided pre-installed or encapsulated in the delivery system 44. The delivery system 44 may comprise a delivery sheath containing the implant. The delivery sheath may be configured (e.g., shaped and/or dimensioned) to be inserted into the deployment position via the anterior chamber of the eye. The delivery system 44 may be configured such that the delivery sheath can be withdrawn from the eye while leaving the implant within the eye.

The delivery system 44 may comprise a handle 46 and a retrieval mechanism 48. The retrieval mechanism 48 comprises a gear and rack configured to drive relative movement between a delivery sheath and an implant inside the delivery sheath. A core member comprising a wire or tube may be provided inside the delivery sheath to allow the delivery sheath to be withdrawn without a corresponding movement of the implant. In an embodiment, a lumen of the delivery sheath is smaller than a radius of the implant when the anchoring structure 26 is in a radially expanded state such that the anchoring structure 26 is constrained to be in a radially contracted state when in the delivery sheath. A size of the core member is defined by the size of the delivery sheath. After insertion into the anterior chamber, a distal end of the delivery sheath is advanced to the deployment position. The retrieval mechanism 48 is then actuated to pull the delivery sheath backwards relative to the body 22 while the body 22 is maintained at the same position by the core member, thereby deploying the body 22. After the implant is fully deployed, the core member and sheath may be withdrawn from the suprachoroidal space.

In some embodiments, a biodissolvable material (e.g. suture) is provided on or in the body 22. The biodissolvable material may be provided as a coating and/or may be drug-eluting. The biodissolvable material may be provided on the inside and/or the outside of any or all of the conduit structure 24, portion 42 and/or anchoring structure 26. The biodissolvable material may be configured to dissolve progressively over a period of time after deployment and to thereby gradually alter an effect of the implant on flow of aqueous humour through the conduit from the anterior chamber to the subconjunctival space. The gradual alteration may, for example, comprise a gradual increase in the flow (i.e., a gradual decrease in outflow resistance). Configuring the implant to cause the outflow to increase gradually in this way (rather than suddenly) may decrease the risk of early hypotony. The gradual alternation may occur over a period of time of at least 1 day, optionally at least 1week, optionally at least 2 weeks, optionally at least 4 weeks, optionally at least 2 months, optionally at least 6 months.

In an embodiment, the biodissolvable material is provided inside a portion of the body, such as inside the conduit (or inner lumen) defined by the conduit structure 24, such that the progressive dissolving of the biodissolvable material progressively unblocks the conduit. Alternatively or additionally, the biodissolvable material may be configured to mechanically constrain the anchoring structure 26 such that the progressive dissolving of the biodissolvable material causes the anchoring structure to progressively expand (as the constraining effect is gradually relaxed as the biodissolvable material thins and becomes mechanically weaker). FIGS. 30 and 31 schematically depict provision of a biodissolvable material as a coating 50 over anchoring structures 26 of the type depicted schematically in FIGS. 9 and 17 respectively. The coating 50 provides a webbing or membrane that extends between individual arms 28 of the anchoring structures 26, for example like an umbrella. The coating 50 in the form of a membrane can be impermeable or porous. The membrane may be provided in the form of a mesh. As the coating dissolves the arms 28 are gradually allowed to extend further outwards in the radial direction. In other embodiments, the membrane may be configured to remain in place permanently (i.e., to be non-biodissolvable).

In some embodiments, a coating 50 is provided that increases a smoothness of outer surfaces of the body 22. The coating 50 may coat the arms 28 of the anchoring structure 26 without necessarily spanning between the arms 28. The coating 50 may thus be provided in a way that maintains the porosity of the anchoring structure 26. The coating 50 may also be applied to the conduit structure 24 in a way that maintains the porosity of the conduit structure 24. Alternatively, the coating 50 may be applied such that the conduit structure 24 and/or anchoring structure 26 become impermeable due to the coating 50 (e.g., the coated spans across lateral openings in the body 22). Such an impermeable coating can be applied on the radial inside of any part of the body 22 and/or on the radial outside of any part of the body 22. When such a coating is applied on the inside or outside of the body 22, this may allow the outflow resistance to be controlled particularly accurately because it is easier to predict the flow characteristics of a solid impermeable tube in comparison to the case of a tube having lateral openings.

In some embodiments, the body 22 may comprise a biocompatible dissolvable suture that holds the anchoring structure 26 in the radially contracted state before and during delivery of the body 22 to the deployment position, which may facilitate deployment. As described above, the biocompatible dissolvable suture may be configured to allow the size of the anchoring structure to increase gradually at a subsequent time to controllably apply the IOP reduction and reduce the risk of early hypotony.

Methods may be provided for deploying an implant in accordance with any of the embodiments described herein. The methods may comprise inserting the implant into the eye and positioning the implant at the deployment position. The methods may optionally be augmented with intraoperative application or injection of therapeutic compositions, such as mitomycin C (MMC).

1. An ocular implant comprising:
an elongate body configured to be deployable at a deployment position at which the body extends from the anterior chamber of an eye to the subconjunctival space of the eye, wherein:
the body comprises a conduit structure defining a conduit for promoting flow of aqueous humour through the conduit from the anterior chamber to the subconjunctival space; and
the body comprises an anchoring structure configured to expand from a radially contracted state to a radially expanded state.

2. The implant of claim 1, wherein the anchoring structure is configured to promote formation of a bleb, and/or to push tissue away from an axis of the anchoring structure, in the subconjunctival space when the body is deployed at the deployment position and the anchoring structure is in the radially expanded state.

3. The implant of claim 1, wherein the anchoring structure is configured to expand from the radially contracted state to the radially expanded state substantially without expansion of the conduit structure.

4. The implant of claim 1, wherein the anchoring structure is configured to self-expand and/or be caused to expand by application of a stimulus, optionally by balloon actuation, hydraulic actuation, temperature actuation, or magnetic actuation.

5. The implant of claim 1, wherein the body is configured to be insertable to the deployment position with the anchoring structure in the radially contracted state by applying a radially constraining force to the body during the insertion, and to be deployable at the deployment position by releasing the radially constraining force applied to the body to allow the anchoring structure to self-expand.

6. The implant of claim 1, wherein the conduit defined by the conduit structure has a substantially constant cross-sectional area along a length of the conduit.

7. The implant of claim 1, wherein the conduit is configured to provide an outflow resistance when deployed that can control intraocular pressure to be maintained in the range of about 4 to 20 mm Hg, preferably in the range of about 6 to 12 mm Hg.

8. The implant of claim 1, wherein the anchoring structure is configured to have a maximum radial diameter in the range of about 0.2 mm to about 3.5 mm when deployed at the deployment position in the radially expanded state.

9. The implant of claim 1, wherein the body is manufacturable by a manufacturing process comprising removing material from a hollow cylindrical tube in a region corresponding to the conduit structure and/or in a region corresponding to the anchoring structure.

10. The implant of claim 9, wherein the removal of material from the region corresponding to the conduit structure increases a flexibility of the conduit structure, optionally forming a spiral.

11. The implant of claim 9, wherein the manufacturing process comprises deforming the tube in the region corresponding to the anchoring structure, after the removal of material in the region, to define the anchoring structure and configure the anchoring structure to self-expand from the radially contracted state to the radially expanded state.

12. The implant of claim 1, wherein the conduit structure has a generally cylindrical form in a relaxed state and defines one or more lateral openings configured to facilitate bending of the conduit structure about axes perpendicular to a longitudinal axis of the conduit structure.

13. The implant of claim 1, wherein the anchoring structure comprises a plurality of arms, at least a subset of which extend along paths that each lie within a different respective plane containing a longitudinal axis of the anchoring structure.

14. The implant of claim 13, wherein the arms are arranged in a rotationally asymmetric manner to promote rotationally asymmetric pushing away of tissue in the region of the anchoring structure when the body is deployed at the deployment position.

15. The implant of claim 13, wherein the arms are configured such that, when the anchoring structure is in the radially expanded state, the arms radially diverge as a function of position towards a distal tip of the body along a first portion of the longitudinal axis of the anchoring structure.

16. The implant of claim 15, wherein at least two of the arms have different maximum radial divergences.

17. The implant of claim 16, wherein two or more of the arms have the same maximum radial divergence and a single one of the arms has a lower maximum radial divergence.

18. The implant of claim 15, wherein the plurality of arms comprises a first pair of arms extending along paths that lie in a same first plane and a second pair of arms extending along paths that lie in a same second plane, the first and second planes optionally being orthogonal to each other.

19. The implant of claim 15, wherein, when the anchoring structure is in the radially expanded state, the plurality of arms radially converge as a function of position towards the distal tip of the body along a second portion of the longitudinal axis of the anchoring structure, the first portion being between the conduit structure and the second portion.

20. The implant of claim 13, wherein at least two of the arms converge and join at or near a distal tip of the anchoring structure.

21. The implant of claim 13, wherein at least two of the arms are disconnected from each other at distal extremities of the arms.

22. The implant of claim 1, wherein the body is configured such that when the body is in a relaxed state before deployment a longitudinal axis of the conduit structure is coaxial with a longitudinal axis of the anchoring structure.

23. The implant of claim 1, wherein the body is configured such that when the body is in a relaxed state before deployment a longitudinal axis of the conduit structure is aligned obliquely with respect to a longitudinal axis of the anchoring structure.

24. The implant of claim 1, comprising a biodissolvable material on or in the body, the biodissolvable material being configured to dissolve progressively over a period of time after deployment and to thereby gradually alter an effect of the implant on flow of aqueous humour through the conduit defined by the conduit structure from the anterior chamber to the subconjunctival space.

25. The implant of claim 24, wherein the biodissolvable material is provided inside a portion of the body such that the progressive dissolving of the biodissolvable material progressively unblocks the conduit.

26. The implant of claim 24, wherein the biodissolvable material is configured to mechanically constrain the anchoring structure such that the progressive dissolving of the biodissolvable material causes the anchoring structure to progressively expand.

27. A kit for deploying an implant into an eye, comprising:
the implant of claim 1; and
a delivery system configured to deliver the implant to the deployment position.

28. A method of deploying the implant of claim 1, the method comprising inserting the implant into the eye and positioning the implant at the deployment position.
